(19) United States
(12) Patent Application Publication
(10) Pub. No.: US 2024/0043493 A1
(43) Pub. Date: Feb. 8, 2024

(54) ACYLATED SINGLE-CHAIN INSULIN ANALOGUES

(71) Applicant: CASE WESTERN RESERVE UNIVERSITY, Cleveland, OH (US)

(72) Inventors: Michael A. WEISS, Indianapolis, IN (US); Nelson B. PHILLIPS, Seven Hills, OH (US); Faramarz ISMAIL-BEIGI, Shaker Heights, OH (US)

(21) Appl. No.: 18/265,616

(22) PCT Filed: Dec. 7, 2021
(86) PCT No.: PCT/US21/62267

§ 371 Date: Jun. 6, 2023

Related U.S. Application Data
(60) Provisional application No. 63/122,373, filed on Dec. 7, 2020.

(51) Int. Cl. C07K 14/62; A61P 3/10

(52) U.S. Cl. CPC C07K 14/62; A61P 3/10

(57) ABSTRACT

A single-chain insulin analogue comprises the insulin B-chain polypeptide sequence, the insulin A-chain polypeptide sequence, and a connecting polypeptide sequence of 5-11 amino acids linking the C-terminal amino acid of the B-chain polypeptide to the N-terminal amino acid of the A-chain polypeptide. The analogue comprises an acetylated Lys at a location selected from the group consisting of any of the amino acids in the connecting polypeptide, B0-B3, B28-B29 or A14, relative to wild type insulin, or comprises an acetylated amino acid at the N-terminal amino acid of the single-chain insulin analogue. The single-chain insulin analogue may be acylated with a C₆-C₂₁ fatty acid, which may be attached to the e-amino group of a unique Lysine residue or the a-amino group of the N-terminal amino acid of the single-chain insulin analogue. The insulin analogue may be used to lower the blood sugar of a patient in need thereof.

### CROSS REFERENCE TO RELATED APPLICATIONS

This application claims benefit of pending U.S. Provisional Application No. 63/122,373 filed on Dec. 7, 2020, the contents of which are incorporated by reference herein.

### STATEMENT REGARDING FEDERALLY SPONSORED RESEARCH OR DEVELOPMENT

This invention was made with government support under grant numbers DK040949 and DK074176 awarded by the National Institutes of Health. The government has certain rights in the invention.

### BACKGROUND OF THE INVENTION

This invention relates to polypeptide hormone analogues that exhibits enhanced pharmaceutical properties, such as increased thermodynamic stability, augmented resistance to thermal fibrillation above room temperature, decreased mitogenicity, and/or altered pharmacokinetic and pharmacodynamic properties, i.e., conferring more prolonged duration of action or more rapid duration of action relative to soluble formulations of the corresponding wild-type human hormone. More particularly, this invention relates to acylated insulin analogues consisting of a single polypeptide chain that contains a novel class of foreshortened connecting (C) domains between A and B domains. The purpose of the acylation is to confer a protracted pharmacokinetic profile—believed to be due to albumin binding and possible stabilization of the subcutaneous depot—leading to an extended pharmacodynamic profile relative to a similar but un-acetylated analogue. Such an extended profile would confer therapeutic benefits in the treatment of patients with diabetes mellitus in a class of insulin analogues remarkable for their resistance to physical degradation at or above room temperature.

The engineering of non-standard proteins, including therapeutic agents and vaccines, may have broad medical and societal benefits. Naturally occurring proteins—as encoded in the genomes of human beings, other mammals, vertebrate organisms, invertebrate organisms, or eukaryotic cells in general—often confer multiple biological activities. A benefit of non-standard proteins would be to achieve selective activity, such as decreased binding to homologous cellular receptors associated with an unintended and unfavorable side effect, such as promotion of the growth of cancer cells. Yet another example of a societal benefit would be augmented resistance to degradation at or above room temperature, facilitating transport, distribution, and use.

An example of a therapeutic protein is provided by insulin. Wild-type human insulin and insulin molecules encoded in the genomes of other mammals bind to insulin receptors is multiple organs and diverse types of cells, irrespective of the receptor isoform generated by alternative modes of RNA splicing or by alternative patterns of post-translational glycosylation. Wild-type insulin also binds with lower affinity to the homologous Type 1 insulin-like growth factor receptor (IGF-1R).

An example of a further medical benefit would be optimization of the stability of a protein regarding unfolding or degradation. Such a societal benefit would be enhanced by the engineering of proteins more refractory than standard proteins with respect to degradation at or above room temperature for use in regions of the developing world where electricity and refrigeration are not consistently available. Analogues of insulin consisting of a single polypeptide chain and optionally containing non-standard amino-acid substitutions may exhibit superior properties with respect to resistance to thermal degradation or decreased mitogenicity. The challenge posed by its physical degradation is deepened by the pending epidemic of diabetes mellitus in Africa and Asia. Because fibrillation poses the major route of degradation above room temperature, the design of fibrillation-resistant formulations may enhance the safety and efficacy of insulin replacement therapy in such challenged regions.

Administration of insulin has long been established as a treatment for diabetes mellitus. A major goal of conventional insulin replacement therapy in patients with diabetes mellitus is tight control of the blood glucose concentration to prevent its excursion above or below the normal range characteristic of healthy human subjects. Excursions below the normal range are associated with immediate adrenergic or neuroglycopenic symptoms, which in severe episodes lead to convulsions, coma, and death. Excursions above the normal range are associated with increased long-term risk of microvascular disease, including retinopathy, blindness, and renal failure.

Insulin is a small globular protein that plays a central role in metabolism in vertebrates. Insulin contains two chains, an A chain, containing 21 residues, and a B chain containing 30 residues. The hormone is stored in the pancreatic β-cell as a Zn²⁺-stabilized hexamer, but functions as a Zn²⁺-free monomer in the bloodstream. Insulin is the product of a single-chain precursor, proinsulin, in which a connecting region (35 amino acid residues) links the C-terminal residue of B chain (residue B30) to the N-terminal residue of the A chain (FIG. 1A). A variety of evidence indicates that it consists of an insulin-like core and disordered connecting peptide (FIG. 1B). Formation of three specific disulfide bridges (A6-A11, A7-B7, and A20-B19; FIGS. 1A and 1B) is thought to be coupled to oxidative folding of proinsulin in the rough endoplasmic reticulum (ER). Proinsulin assembles to form soluble Zn²⁺-coordinated hexamers shortly after export from ER to the Golgi apparatus. Endoproteolytic digestion and conversion to insulin occurs in immature secretory granules followed by morphological condensation. Crystalline arrays of zinc insulin hexamers within mature storage granules have been visualized by electron microscopy (EM). The sequence of insulin is shown in schematic form in FIG. 1C. Individual residues are indicated by the identity of the amino acid (typically using a standard three-letter code), the chain and sequence position (typically as a superscript). Pertinent to the present invention is the invention of novel foreshortened C domains of length 5-11 residues in place of the 36-residue wild-type C domain characteristic of human proinsulin.

Fibrillation, which is a serious concern in the manufacture, storage and use of insulin and insulin analogues for the treatment of diabetes mellitus, is enhanced with higher temperature, lower pH, agitation, or the presence of urea, guanidine, ethanol co-solvent, or hydrophobic surfaces. Current US drug regulations demand that insulin be discarded if fibrillation occurs at a level of one percent or more. Because fibrillation is enhanced at higher temperatures, patients with diabetes mellitus optimally must keep insulin refrigerated prior to use. Fibrillation of insulin or an insulin analogue can be a particular concern for such patients utilizing an external insulin pump, in which small amounts of insulin or insulin analogue are injected into the patient's body at regular intervals. In such a usage, the insulin or insulin analogue is not kept refrigerated within the pump apparatus, and fibrillation of insulin can result in blockage of the catheter used to inject insulin or insulin analogue into the body, potentially resulting in unpredictable fluctuations in blood glucose levels or even dangerous hyperglycemia. Insulin exhibits an increase in degradation rate of 10-fold or more for each 10° C. increment in temperature above 25° C.; accordingly, guidelines call for storage at temperatures <30° C. and preferably with refrigeration. Fibrillation of basal insulin analogues formulated as soluble solutions at pH less than 5 (such as Lantus® (Sanofi-Aventis), which contains an unbuffered solution of insulin glargine and zinc ions at pH 4.0) also can limit their half-lives due to physical degradation at or above room temperature; the acidic conditions employed in such formulations impairs insulin self-assembly and weakens the binding of zinc ions, reducing the extent to which the insulin analogues can be protected by sequestration within zinc-protein assemblies.

Insulin is susceptible to chemical degradation, involving the breakage of chemical bonds with loss or rearrangement of atoms within the molecule or the formation of chemical bonds between different insulin molecules. Such changes in chemical bonds are ordinarily mediated in the unfolded state of the protein, and so modifications of insulin that augment its thermodynamic stability also are likely to delay or prevent chemical degradation.

Insulin is also susceptible to physical degradation. The present theory of protein fibrillation posits that the mechanism of fibrillation proceeds via a partially folded intermediate state, which in turn aggregates to form an amyloidogenic nucleus. In this theory, it is possible that amino-acid substitutions that stabilize the native state may or may not stabilize the partially folded intermediate state and may or may not increase (or decrease) the free-energy barrier between the native state and the intermediate state. Therefore, the current theory indicates that the tendency of a given amino-acid substitution in the two-chain insulin molecule to increase or decrease the risk of fibrillation is highly unpredictable. Models of the structure of the insulin molecule envisage near-complete unfolding of the three-alpha helices (as seen in the native state) with parallel arrangements of beta-sheets formed successive stacking of B-chains and successive stacking of A-chains; native disulfide pairing between chains and within the A-chain is retained. Such parallel cross-beta sheets require substantial separation between the N-terminus of the A-chain and C-terminus of the B-chain (>30 Å), termini ordinarily in close proximity in the native state of the insulin monomer (<10 Å). Marked resistance to fibrillation of single-chain insulin analogues with foreshortened C-domains is known in the art and thought to reflect a topological incompatibility between the splayed structure of parallel cross-beta sheets in an insulin protofilament and the structure of a single-chain insulin analogue with native disulfide pairing in which the foreshortened C-domain constrains the distance between the N-terminus of the A-chain and C-terminus of the B-chain to be unfavorable in a protofilament.

Single-chain insulin analogues might therefore seem to provide a favorable approach toward the design of fibrillation-resistant insulin analogues. However, such analogues often exhibit low activities, which can be 1% or lower relative to wild-type human insulin. (Although Lee, H. C., et al. (2000) claimed that single-chain insulin analogues with wild-type A- and B-domains of length 57 residues or 58 residues exhibit receptor-binding affinities in the range 30-40% relative to human insulin, this publication was retracted in 2009 due to scientific misconduct; in our hands the analogues disclosed by Lee, H. C. et al. exhibit relative affinities of less than 1%.) Affinity might in part be restored by introduction of Asp^{B10}, a substitution known in the art to enhance the affinity of insulin for the insulin receptor. We have previously described a 57-residue single-chain insulin containing Asp^{B10} with C-domain linker GGGPRR. A single-chain insulin analogue with high receptor-binding affinity was described in which the foreshortened C-domain was the 12-residue C-domain of insulin-like growth factor I (IGF-I; sequence GYGSSSRRAPQT), yielding a chimeric protein. However, such chimeric molecules exhibit enhanced relative and absolute affinities for IGF-1R. Such alterations, like those associated with Asp^{B10} and other substitutions at position B10, have elicited broad concern due to possible association with an increased risk of cancer in animals or human patients taking such analogues. This concern is especially marked with respect to basal insulin analogs, i.e., those designed for once-a-day administration with 12-24 hour profile of insulin absorption from a subcutaneous depot and 12-24 hour profile of insulin action.

The present invention was motivated by the medical and societal needs to engineer a basal once-a-day or once-a-week single-chain insulin analogue that combines (i) resistance to degradation with (ii) substantial in vivo hypoglycemic potency with (iii) reduced cross-binding to IGF-1R and (iv) a time-course of action upon subcutaneous injection that is extended by acylation of the single-chain insulin molecule.

It would be desirable, therefore, to invent acylated single-chain insulin analogues with (a) mitogenicity and cross-binding to the IGF-1R that is no higher than that of wild-type human insulin and (b) at least a portion of the glucose-lowering effect of wild-type insulin. It would be also be desirable for the acylated single-chain analogues be formulatable as a clear aqueous solution at either neutral pH (with protein isoelectric point similar to that of wild-type human insulin) or acidic pH (with protein isoelectric point shifted to the range 6.5-7.5) such that protein aggregation occurs at the neutral pH of the subcutaneous depot. More generally, there is a need for an insulin analogue that displays increased thermodynamic stability and increased resistance to fibrillation above room temperature while exhibiting prolonged pharmacokinetic and pharmacodynamic properties.

### SUMMARY OF THE INVENTION

It is, therefore, an aspect of the present invention to provide single-chain insulin analogues that provide decreased cross-binding to IGF-1R and prolonged duration of action while retaining at least a portion of the glucose-lowering activity of wild-type insulin in rodents following subcutaneous injection. It is an additional aspect of the present invention that absolute in vitro affinities of the single-chain insulin analogue for IR-A and IR-B are in the range 1-100% relative to wild-type human insulin and so unlikely to exhibit prolonged residence times in the hormone-receptor complex. It is a further aspect to provide the use of modified single-chain insulin analogues containing a tethered fatty acid or dicarboxylic acid. While not wishing to condition patentability on the operation of any particular theory, the safety and effectiveness of such therapy could in principle be enhanced through increased binding to albumin and may in addition stabilize the subcutaneous depot.

In general, the present invention provides a single-chain insulin analogue comprising the insulin B-chain polypeptide sequence, the insulin A-chain polypeptide sequence, and a connecting polypeptide sequence of 5-11 amino acids linking the C-terminal amino acid of the B-chain polypeptide to the N-terminal amino acid of the A-chain polypeptide. The single chain insulin analogue comprises an acetylated Lys at a location selected from the group consisting of any of the amino acids in the connecting polypeptide, B0-B3, B28-B29 or A14, relative to wild type insulin, or an acetylated amino acid at the N-terminal amino acid of the single-chain insulin analogue. The above combination of features may also include a novel connecting polypeptide or C-domain design wherein a foreshortened connecting polypeptide (length 5-11 residues) contains an N-terminal acidic element (residues C1 and/or C2), a flexible joint or hinge (C3), and C-terminal segment that may be derived from the C-domain of IGF-II (C4-Cₙ, where n=5, 6, 7, 8, 9, 10, or 11). For example, one or both of the two amino acids closest to the C-terminus of the connecting polypeptide may be Arg.

The N-terminal acidic element was designed in accordance with studies of two-chain insulin analogues containing 32-residue B-chains wherein the charges of the basic Arg^{B31}-Arg^{B32} element of insulin glargine were reversed (U.S. Pat. No. 8,399,407, entitled “Non-Standard Insulin Analogues,” published Mar. 31, 2011; incorporated herein by reference). An upper limit of 11 for the C-domain length was chosen to be below the 12-residue IGF-I-derived linker described in a chimeric insulin analogue with enhanced IGF-1R-binding activity. A lower limit of 5 was chosen to enable sufficient play to bind to the insulin receptor with displacement of the B24-B27 segment (as visualized in crystallographic studies of model ectodomain-insulin complexes). Although not wishing to be constrained by theory, we believe that a one- or two-residue acidic residues introduce unfavorable electrostatic repulsion on binding of the analogue to IGF-1R but is well tolerated by insulin receptor isoforms. Also without wishing to be constrained by theory, we further believe that the IGF-II-derived C-terminal segment of the C-domain, an optional element of the present invention, introduces favorable interactions with insulin receptor isoforms and so functions as an ancillary receptor-binding element rather than a mere tether or space element. Receptor-binding affinities lower than that of wild-type insulin may be desirable to delay clearance of the acylated single-chain analogue from the blood stream. The C domains of this class may optionally contain O-linked glycosylation of serine in the C domain. The single-chain insulin analogues of the present invention may optionally contain standard or non-standard amino-acid substitutions at other sites in the A or B domains.

The analogues of the present invention may also contain Histidine at position B10 and so circumvent concerns regarding carcinogenesis that is associated with an acidic substitution (Aspartic Acid or Glutamic Acid) at this position.

The present invention highlights the utility of chemical modification of single-chain insulin analogues by a fatty acid or dicarboxylic acid, optionally linked to the protein molecule via a space element containing 3-30 atoms. The term “fatty acid” designates a saturated or unsaturated C₆-C₂₁ fatty acid or dicarboxylic acid. The preferred fatty acids are saturated and include myristic acid (C₁₄), pentadecylic acid (C₁₅), palmitic acid (C₁₆), heptadecylic acid (C₁₇), stearic acid (C₁₈) and arachidic acid (C₂₀). In one particular example, the fatty acid is palmitic acid. In another example, the fatty acid is arachidic acid.

The compounds of the present invention represent mono-acylated single-chain insulin analogues. The insulin analogues are acylated at an α-amino group or ε-amino group with a C₆-C₂₁ fatty acid or dicarboxylic acid. The analogues may be mono-acylated at either B1 or ε-amino group of a unique lysine at one of the following positions: B3, B28, B29, A14 or within a foreshortened C domain (5-11 residues in length). The term “activated fatty acid ester” or “activated dicarboxylic acid ester” designates a fatty acid which has been activated at one end using general techniques described in Methods of Enzymology 25: 494-499 (1972) and/or Lapidot et al., in J. of Lipid Res. 8: 142-145 (1967). Activated fatty acid esterinclude derivatives of commonly employed acylating agents such as hydroxybeozotriazide (HOBT), N-hydroxysuccininlide and related derivatives. One particular activated ester is N-succinimidyl palmitate. The term “soluble” indicates that a sufficient amount of ester is present in the liquid phase to acylate the insulin analogue as would typically be conferred by 1 to 2 molar equivalents of activated ester per mole of analogue are in the liquid phase.

The general formula for a dicarboxylic acid is HO₂C(CH₂)ₙCO₂H. In the present invention on carboxylate group would be derivatized for attachment to a nitrogen atom in the single-chain insulin analog, and the other carboxylate group would be exposed to solvent. The intervening methylene chain would mediate binding to albumin and possibly to other analogue molecules or components of the subcutaneous space. Examples are provided by subacic acid (n=8 in the above general formula; decanedioic acid), dodecanedioic acid (n=10), and thapsic acid (n=14; hexadecanedioic acid). Dicarboxic acids containing n methylene groups thus contain n+2 carbon atoms. An example of a spacer element is provided by γ-L-glutamyl element as known in the art in the insulin product insulin degludec (Trisiba; Novo-Nordisk). This spacer element contains 16 atoms as an intervening element (excluding one oxygen and one hydrogen in the free molecule of γ-L-glutamate as a zwitterion at neutral pH).

The single-chain insulin analogues of the present invention fall into two classes based on point of attachment: either via (i) the unique N-terminal α-amino group or (ii) the s-amino group of a unique Lysine. We qualify these chemical classes as follows. (i) The N-terminal α-amino group may be at position B1, at a position “B0” upon introduction of an N-terminal extension of the B domain, or upon successive N-terminal deletion of the B domain, at neo-N-terminal positions B2, or B3. In case of N-terminal deletion the neo-N-terminal residue and following residue may optionally be substituted to optimize chemical stability. (ii) The unique Lysine residue may be positioned at one of the following positions: B0, B3, B28, B29, A8 or A14; additionally, a unique Lysine may be introduced within the foreshortened C domain.

The above family of acylated single-chain insulin analogues may also be classified according to protein isoelectric point and hence preferred pH of pharmaceutical formulation. The first pI-defined class consists of analogues whose isoelectric point is less than 6.0; such single-chain analogues may be formulated as a clear, soluble solution at pH 7.4. The second pI-defined class consists of analogues whose isoelectric point is between 6.5 and 7.5; such single-chain analogues may be formulated as a clear, soluble solution in the pH range 3.0-4.0. The latter class would exhibit two distinct mechanisms of protracted action based on isoelectric precipitation in the subcutaneous depot and a combination of acyl-mediated albumin binding and potential acyl-mediated stabilization of the subcutaneous depot.

### BRIEF DESCRIPTION OF THE SEVERAL VIEWS OF THE DRAWINGS

FIG. 1A is a schematic representation of the sequence of human proinsulin including the A- and B domains and the connecting region shown with flanking dibasic cleavage sites (filled circles) and C peptide (open circles).

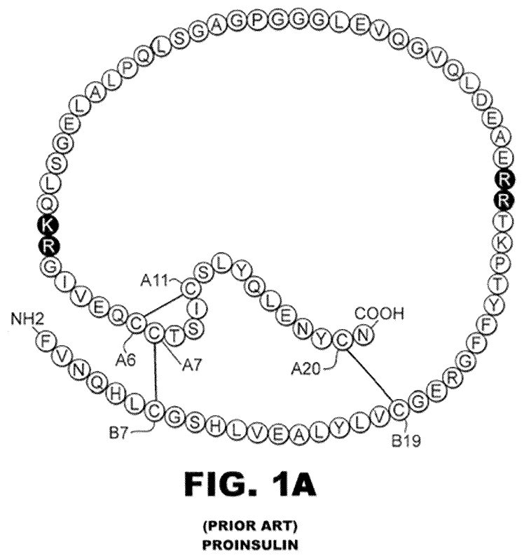

FIG. 1B is a structural model of proinsulin, consisting of an insulin-like moiety and a disordered connecting peptide (dashed line).

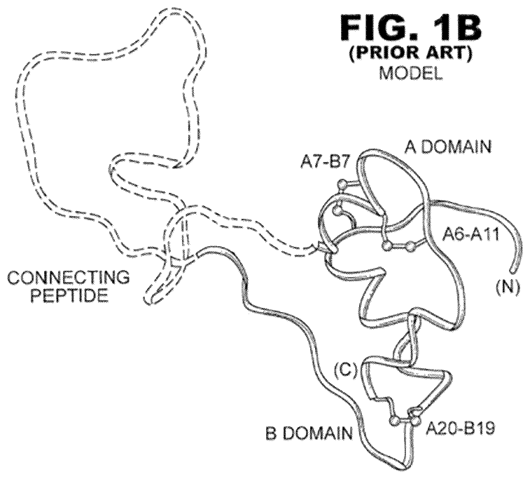

FIG. 1C is a schematic representation of the sequence of human insulin indicating the position of residues B27 and B30 in the B chain.

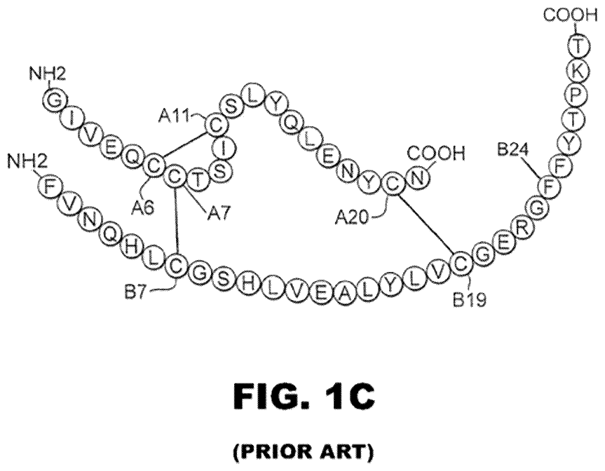

FIG. 2 is a graph showing the results of rat studies of two-chain and single-chain insulin analogues in animals rendered diabetic by treatment with the β-cell toxin streptozotocin. Data are plotted with respect to blood-glucose concentration (vertical scale in panel A) or as a fraction of the initial blood-glucose concentration (B). Symbols are defined under the panels. Results demonstrate marked prolongation of a representative single-chain insulin analogue upon modification of the α-amino group of PheB1 by palmitic acid.

### DETAILED DESCRIPTION OF THE INVENTION

It is also envisioned that single-chain analogues may also be made with A- and B domain sequences derived from animal insulins, such as porcine, bovine, equine, and canine insulins, by way of non-limiting examples. In addition or in the alternative, the insulin analogue of the present invention may contain a deletion of residue B1, residues B1-B2 or residues B1-B3, optionally with amino-acid substitutions of the neo-N-terminal residue and the following residue to enhance chemical stability. An example of such a neo-N-terminal sequence is provided by des-B1, AlaB2 and GluB3. The B domain may also be extended by a “residue B0,” envisioned as a space element such that the α-amino group is displaced by residue B1 by three atoms; an example of an optional residue B0 is provided by Alanine or Glutamic Acid, by way of non-limiting examples.

The single-chain insulin analogues of the present invention may contain a unique Lysine residue, either at position B29 (as in wild-type human insulin) or at positions B3, B28, A8, A14 or within the C domain; in the latter embodiments the native Ly sine at position B29 would be substituted by Alanine, Glutamic Acid or Proline. In the case of Proline at position B29, the native ProB28 would be substituted to avoid a Pro-Pro element at positions B28-B29.

The A domains of the present invention may also contain substitutions at position A21 to avoid deamidation of the native Asparagine and other pathways of chemical degradation; examples of substitutions at A21 are provided by Gly, Ala, Ser, Thr and Glu.

The B-domain of the single-chain insulin of the present invention may optionally contain non-standard substitutions, such as D-amino-acids at positions B20 and/or B23 (intended to augment thermodynamic stability, receptor-binding affinity, and resistance to fibrillation), a halogen modification at the 2 ring position of Phe^{B24} (i.e., ortho_F_Phe^{B24}, ortho-Cl-Phe^{B24}, or ortho-Br-Phe^{B24}; intended to enhance thermodynamic stability and resistance to fibrillation), 2-methyl ring modification of Phe^{B24} (intended to enhance receptor-binding affinity), and/or introduction of iodo-substitutions within the aromatic ring of Tyr^{B16} and/or Tyr^{B26} (3-mono-iodo-Tyr or [3, 5]-di-iodo-Tyr); intended to augment thermodynamic stability and receptor-binding activity). It is also envisioned that Thr^{B27}, Thr^{B30}, or one or more Serine residues in the C-domain may be modified, singly or in combination, by a monosaccharide adduct; examples are provided by O-linked N-acetyl-β-D-galactopyrano side (designated GalNAc-O^{β}-Ser or GalNAc-O^{β}-Thr), O-linked α-D-mannopyrano side (mannose-O^{β}-Ser or mannose-O^{β}-Thr), and/or α-D-glucopyranoside (glucose-O^{β}-Ser or glucose-O^{β}-Thr). Furthermore, in view of the similarity between human and animal insulins, and use in the past of animal insulins in human patients with diabetes mellitus, it is also envisioned that other minor modifications in the sequence of insulin may be introduced, especially those substitutions considered “conservative.” For example, additional substitutions of amino acids may be made within groups of amino acids with similar side chains, without departing from the present invention. These include the neutral hydrophobic amino acids: Alanine (Ala or A), Valine (Val or V), Leucine (Leu or L), Isoleucine (Ile or I), Proline (Pro or P), Tryptophan (Trp or W), Phenylalanine (Phe or F) and Methionine (Met or M). Likewise, the neutral polar amino acids may be substituted for each other within their group of Glycine (Gly or G), Serine(Ser or S), Threonine (Thr or T), Tyrosine (Tyr or Y), Cysteine (Cys or C), Glutamine (Glu or Q), and Asparagine (Asn or N). Basic amino acids are considered to include Lysine (Lys or K), Arginine (Arg or R) and Histidine (His or H). Acidic amino acids are Aspartic acid (Asp or D) and Glutamic acid (Glu or E). Unless noted otherwise or wherever obvious from the context, the amino acids noted herein should be considered to be L-amino acids. Standard amino acids may also be substituted by non-standard amino acids belong to the same chemical class. By way of non-limiting example, the basic side chain Lys may be replaced by basic amino acids of shorter side-chain length (Ornithine, Diaminobutyric acid, or Diaminopropionic acid). Lys may also be replaced by the neutral aliphatic isostere Norleucine (Nle), which may in turn be substituted by analogues containing shorter aliphatic side chains (Aminobutyric acid or Aminopropionic acid).

The amino-acid sequence of human proinsulin is provided, for comparative purposes, as SEQ ID NO: 1.

(human proinsulin)
SEQ ID NO: 1
Phe-Val-Asn-Gln-His-Leu-Cys-Gly-Ser-His-Leu-
Val-Glu-Ala-Leu-Tyr-Leu-Val-Cys-Gly-Glu-Arg-
Gly-Phe-Phe-Tyr-Thr-Pro-Lys-Thr-Arg-Arg-Glu-
Ala-Glu-Asp-Leu-Gln-Val-Gly-Gln-Val-Glu-Leu-
Gly-Gly-Gly-Pro-Gly-Ala-Gly-Ser-Leu-Gln-Pro-
Leu-Ala-Leu-Glu-Gly-Ser-Leu-Gln-Lys-Arg-Gly-
Ile-Val-Glu-Gln-Cys-Cys-Thr-Ser-Ile-Cys-Ser-
Leu-Tyr-Gln-Leu-Glu-Asn-Tyr-Cys-Asn

The amino-acid sequence of the A chain of human insulin is provided as SEQ ID NO: 2.

(human A chain)
SEQ ID NO: 2
Gly-Ile-Val-Glu-Gln-Cys-Cys-Thr-Ser-Ile-Cys-
Ser-Leu-Tyr-Gln-Leu-Glu-Asn-Tyr-Cys-Asn

The amino-acid sequence of the B chain of human insulin is provided as SEQ ID NO: 3.

(human B chain)
SEQ ID NO: 3
Phe-Val-Asn-Gln-His-Leu-Cys-Gly-Ser-His-Leu-
Val-Glu-Ala-Leu-Tyr-Leu-Val-Cys-Gly-Glu-Arg-
Gly-Phe-Phe-Tyr-Thr-Pro-Lys-Thr

The amino-acid sequence of a representative single-chain insulin analogue without a Lysine residue that is suitable for acylation of the α-amino group and whose isoelectric point similar or lower than that of wild-type insulin is given in SEQ ID NO: 4.

SEQ ID NO: 4
Phe-Val-Asn-Gln-His-Leu-Cys-Gly-Ser-His-Leu-
Val-Glu-Ala-Leu-Tyr-Leu-Val-Cys-Gly-Glu-Arg-
Gly-Phe-Phe-Tyr-Thr-Pro-Arg-Thr-Glu-Glu-Gly-
Pro-Xaa₁-Arg-Gly-Ile-Val-Glu-Gln-Cys-Cys-Xaa₂-
Ser-Ile-Cys-Ser-Leu-Xaa₃-Gln-Leu-Glu-Asn-
Tyr-Cys-Xaa₄

Where Xaa₁ indicates Ala, Glu or Arg; Xaa₂ indicates Ala, Glu, Gln, His, or Thr; where Xaa₃ is Tyr or Glu; and where Xaa₄ is Gly, Ala, Asn, Ser or Thr.
Single-chain insulin analogues lacking Lysine at any position are given in SEQ ID NO: 5 such that the isoelectric points of these analogues may either be in the range 4.0-6.0 or in the range 6.5-7.5, depending on the number of acidic and basic residues (as determined following acylation or medication of the targeted amino group).

SEQ ID NO: 5
X₁-Gln-His-Leu-Cys-Gly-Ser-His-Leu-Val-Glu-
Ala-Leu-Tyr-Leu-Val-Cys-Gly-Glu-Arg-Gly-Phe-
Phe-Tyr-Thr-Pro-X₃-Thr-X₂-Gly-Ile-Val-Glu-
Gln-Cys-Cys-X₄-Ser-Ile-Cys-Ser-Leu-X₅-
Gln-Leu-Glu-Asn-Tyr-Cys-X₆

Where X₁ an N-terminal segment containing either four amino acids (residues B0-B3), three amino acids (residues B1-B3), two amino acids (residues B2-B3) or one amino acids (residue B3) such that residue B0, if present, is Ala or Glu, residue B1, if present, is Ala or Phe, residue B2, if present is Ala or Val, and residue B3 is Ala, Asn or Glu; where X₂ is a foreshortened C domain of 5-11 residues such that either residue C1 or C2 is Glu and where the final two residues in the segment contain at least one Arg; where X₃ indicates Ala, Glu or Arg; X₄ indicts Ala, Glu, Gln, His, or Thr; where X₅ is Tyr or Glu; and where X₆ is Gly, Ala, Asn, Ser or Thr.
The single-chain insulin analogues broadly conforming to SEQ ID NO:5 may optionally be further modified to contain a unique Lysine residue as an attachment point for modification by a fatty acid (with optional space element) or dicarboxylic acid (with optional spacer element) at its ε-amino group. This Lys may be introduced at one of the following positions: B3, B28, B29, A8, A14 or within the C domain, provided that if the Lysine is not at native position B29 (as in wild-type human insulin), then residue B29 is substituted by Ala, Glu or Arg. Single-chain insulin analogues containing a unique Lysine at provided here may exhibit isoelectric points either in the range 4.0-6.0 or in the range 6.5-7.5, depending on the number of acidic and basic residues, as determined following acylation or medication of the targeted amino group.

SEQ ID NO: 6
X₁-Gln-His-Leu-Cys-Gly-Ser-His-Leu-Val-Glu-
Ala-Leu-Tyr-Leu-Val-Cys-Gly-Glu-Arg-Gly-
Phe-Phe-Tyr-Thr- X₃-X₄-Thr-X₂-Gly-Ile-Val-
Glu-Gln-Cys-Cys-X₅-Ser-Ile-Cys-Ser-Leu-X₆-
Gln-Leu-Glu-Asn-Tyr-Cys-X₇

Where X₁ an N-terminal segment containing optionally four amino acids (residues B0-B3), three amino acids (residues B1-B3), two amino acids (residues B2-B3) or one amino acids (residue B3) such that residue B0, if present, is Ala or Glu, residue B1, if present, is Ala or Phe, residue B2, if present is Ala or Val, and residue B3 is Ala, Asn, Glu or optionally a unique Lys; where X₂ is a foreshortened C domain of 5-11 residues such that either residue C1 or C2 is Glu, where the final two residues in the segment contain at least one Arg and such that the C segment may optionally contain a unique Lysine; where X₃ indicates Pro, or if B29 is not Lys, then B28 could be Ala, Asp, Glu or optionally the choice of a unique Lys; where X₄ indicates Ala, Glu, Arg, Pro or optionally a unique Lys as in wild-type insulin; X₅ indicates Ala, Glu, Gln, His, or Thr; where X₆ is Tyr or Glu; and where X₇ is Gly, Ala, Asn, Ser or Thr. At positions B28-B29 Pro-Pro is excluded.

To evaluate the biological activity and potency of the analogues in an animal model, male Sprague-Dawley rats (mean body mass ˜300 grams) were rendered diabetic by treatment with streptozotocin (STZ). Protein solutions containing KP-insulin (insulin Lispro, the active component of Humalog®), insulin detemir (Levemir®; Novo-Nordisk), a representative single-chain insulin analogue (with isoelectric point in the range 4.0-6.0), and the same single-chain insulin upon acylation as an embodiment of the present invention. Formulations were based on Lilly diluent (obtained from Eli Lilly and Co.), which is composed of 16 mg glycerin, 1.6 mg meta-cresol, 0.65 mg phenol, and 3.8 mg sodium phosphate pH 7.4. Rats were injected subcutaneously at time t=0 in groups of five (N=5). Blood was obtained from the clipped tip of the tail at time 0 and every 10 minutes up to 360 min. In brief, the acylated analogue of the present invention were found, under conditions of formulation similar to that of Levemir®, to retain a proportion of the biological activity of insulin detemir but with greater duration of action.

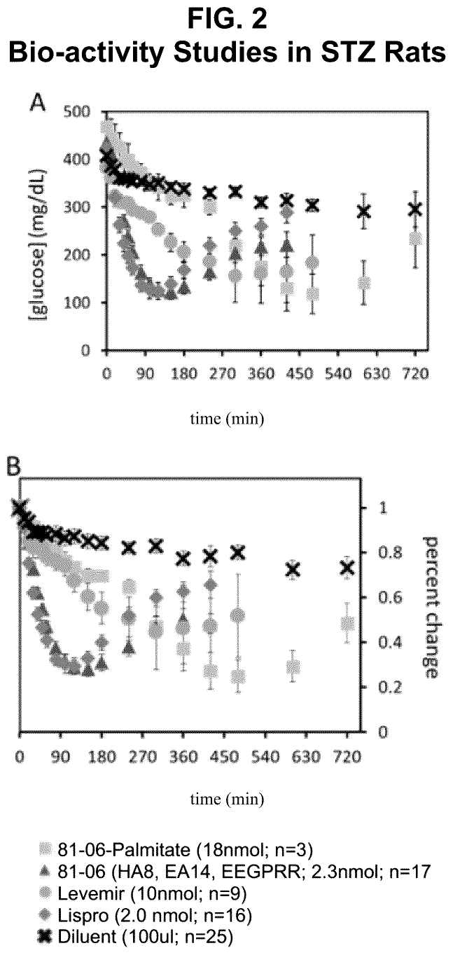

The present invention is directed toward modification of a single-chain insulin analogue by a fatty acid or dicarboxylic acid, optionally with a space element, such that the pharmacokinetic and pharmacodynamic profiles of the analogues are extended relative to the parent unmodified analogues. This idea was reduced to practice by acylation of a representative single-chain insulin analogue by palmitic acid at the α-amino group of PheB1. The analogue belongs to the class defined by an isoelectric point similar to that of wild-type insulin and so amenable to formulation as a clear, soluble solution at neutral pH. It further belongs to the chemical class of sequences lacking a Lysine residue. The sequence of this single-chain insulin analogue conforms to SEQ ID NO: 4 below with Xaa₁ chosen as Glu; Xaa₂ chosen as His; Xaa₃ as Glu; and Xaa₄ as Asn. In this embodiment no spacer element was inserted between the palmitic acid and PheB1. Rat studies of this acylated analogue demonstrated a reduction in intrinsic potency (per nanomole) but with a marked prolongation in the duration of activity (FIG. 2). Control insulins in this figure are provided by insulin detemir (the active component of Levemir®; Novo-Nordisk) and insulin lispro (the active component of Humalog®; Eli Lilly).

Further examples of the claimed invention were also synthesized as follows. Solid-phase peptide synthesis was used to prepare six SCI analogues wherein specific residues were substituted by Lysine (bold in Table 1; the parent SCI is shown in row 5). The parent SCI is related by the amino-acid substitution GluB29→Arg to SCI-a as described in Glidden, M. D. et al. J. Biol. Chem. 293(1):47-68 (2018). We designate the analogues as SCI-a-X in relation to that publication, where X indicates the amino-acid substitution(s).

The variant 57-residue peptides were synthesized starting with pre-loaded H-Asn(Trt)-HMBP-ChemMatrix resin or H-Gly-(E)-4-hydroxy-3-methylbut-2-enyl phosphate (HMBP)-ChemMatrix resins (Protein Technologies) using traditional fluorenylmethoxycarbonyl/tert-butyl (Fmoc/tBu) chemistry with repetitive N,N′-diisopropylcarbodiimide/1-Hydroxy-6-chloro-benzotriazole (DIC/6-Cl-HOBt) or DIC/OxymaPure® activation/coupling cycles (10 equivalents) and IR or induction heating at 60° C. for 10 min per cycle and 50° C. for Fmoc deprotection (20% piperidine/DMF, 2×5 min). OxymaPure® (2-cyano-2-(hydroxyimino)acetate) displays a remarkable capacity to suppress racemization and an impressive coupling efficiency in both automated and manual synthesis. Tribute or Chorus automated peptide synthesizers (Gyros Protein Technology, Tucson, AZ) were used throughout. Amino acids, DIC and 6-Cl-HOBt were purchased from Gyros Protein Technology (Tucson, AZ). Peptides were cleaved from resin and deprotected by treatment (15 ml per 0.1 mmol scale, 4 hrs) with trifluoroacetic acid (TFA) containing 2.5% triisopropylsilane (TIS), 2.5% water, 2.5% DODT (ethylenedioxy-diethanethiol), and 2.5% of anisole.

TABLE 1
Single-Chain Insulins Containing Single
Lysine Substitutions

| Sample | Name | Sequence |
|---|---|---|
| 1 | SCI-a- | FVNQHLCGSHLVEALYLVCGERG |
| | KB29 | FFYTPKTEEGPRRGIVEQCCHSI |
| | | CSLEQLENYCN |
| | | (SEQ ID NO: 7) |
| 2 | SCI-a- | FVNQHLCGSHLVEALYLVCGERG |
| | KB28, | FFYTKPTEEGPRRGIVEQCCHSI |
| | PB29 | CSLEQLENYCN |
| | | (SEQ ID NO: 8) |
| 3 | SCI-a- | FVNQHLCGSHLVEALYLVCGERG |
| | RB29, | FFYTPRTEEGPKRGIVEQCCHSI |
| | KC5 | CSLEQLENYCN |
| | | (SEQ ID NO: 9) |
| 4 | SCI-a- | FVKQHLCGSHLVEALYLVCGERG |
| | KB3, | FFYTPRTEEGPRRGIVEQCCHSI |
| | RB29 | CSLEQLENYCN |
| | | (SEQ ID NO: 10) |
| 5 | SCI-a- | AFVNQHLCGSHLVEALYLVCGER |
| | AB0, | GFFYTPRTEEGPRRGIVEQCCHS |
| | RB29 | ICSLEQLENYCN |
| | | (SEQ ID NO: 11) |
| 6 | SCI-a- | FVNQHLCGSHLVEALYLVCGERG |
| | KA14, | FFYTPRTEEGPRRGIVEQCCHSI |
| | RB29 | CSLKQLENYCN |
| | | (SEQ ID NO: 12) |
| 7 | SCI-a- | FVNQHLCGSHLVEALYLVCGERG |
| | TA8, | FFYTPKTEEGPRRGIVEQCCTSI |
| | GA21, | CSLEQLENYCG |
| | KB29 | (SEQ ID NO: 13) |

Single Chain Insulins (SCIs) (Samples 1-7) were acylated to provide Samples 8-14 by dissolving in aqueous sodium carbonate (0.1M), tetrahydrofuran (THF) buffer (1:1) at 12 mg/ml (approximately 1.8 M) to which was added the N-hydroxysuccinimido (OSnu) activated ester of fatty acids: Myristic acid (C14), PALMITIC ACID (C16), STEARIC ACID (C18), ARACHIDIC ACID (C20). Accordingly, C20-OSnu (5 equivalents,) was dissolved in dimethylacetamide (DMA), tetrahydrofuran (THF) (1:1 at 7.2 mg/ml). The reaction mixture was allowed to react for 10 min or until formation of the mono-acylated SCI was optimum as determined by analytical reversed-phase high-performance liquid chromatography (rp-HPLC). The acylation reaction was quenched to pH 2-3 by addition of HCl (5 N) then diluted five-to-six-fold with guanidine hydrochloride (Gu-HCl 6 M in 0.1% vol/vol trifluoroacetic acid [TFA]) and purified by rp-HPLC. The sequences of the resulting acylated SCI samples are provided in Table 2 with the location of the acylated Lysine indicated with “K(C20)” provided in bold.

TABLE 2
Single-Chain Insulins Containing C20
acylation on Lysine Residues

| Sample | Name | Sequence |
|---|---|---|
| 8 | SCI-a- | FVNQHLCGSHLVEALYLVCGERGFF |
| | KB28,PB29 | YTK(C20)PTEEGPRRGIVEQCCHS |
| | | ICSLEQLENYCN |
| | | (SEQ ID NO: 14) |
| 9 | SCI-a- | FVNQHLCGSHLVEALYLVCGERGF |
| | RB29, | FYTPRTEEGPK(C20)RGIVEQCC |
| | KC5 | HSICSLEQLENYCN |
| | | (SEQ ID NO:15) |
| 10 | SCI-a- | FVK(C20)QHLCGSHLVEALYLVCG |
| | KB3, | ERGFFYTPRTEEGPRRGIVEQCCHSI |
| | RB29 | CSLEQLENYCN (SEQ ID NO:16) |
| 11 | SCI-a- | FVNQHLCGSHLVEALYLVCGERGFF |
| | KB29 | YTPK(C20)TEEGPRRGIVEQCCHS |
| | | ICSLEQLENYCN |
| | | (SEQ ID NO: 17) |
| 12 | SCI-a- | C20- |
| | AB0, | AFVNQHLCGSHLVEALYLVCGERGF |
| | RB29 | FYTPRTEEGPRRGIVEQCCHSI |
| | | CSLEQLENYCN |
| | | (SEQ ID NO: 18) |
| 13 | SCI-a- | FVNQHLCGSHLVEALYLVCGERGF |
| | KA14, | FYTPRTEEGPRRGIVEQCCHSI |
| | RB29 | CSLK(C20)QLENYCN |
| | | (SEQ ID NO: 19) |
| | SCI-a- | FVNQHLCGSHLVEALYLVCGERG |
| 14 | TA8, | FFYTPK(20)TEEGPRRGIVEQC |
| | GA21, | CTSICSLEQLENYCG |
| | KB29 | (SEQ ID NO: 20) |

The acylated SCIs were purified by preparative rp-HPLC on a PROTO 300 C4 (20×250 mm, 10 μm, Higgins Analytical) column with 0.1% TFA/H2O (A) and 0.1% TFA/CH3CN (B) as elution buffers. Identity of the peptides was confirmed by liquid chromatography-mass spectrometry (LC-MS; Finnigan LCQ Advantage) on a TARGA C8 (4.6×250 mm, 5 μm, Higgins Analytical) with 0.1% TFA/H2O (A) and 0.1% TFA/CH3CN as eluents. Acylated SCIs were analyzed using a 2% B per min gradient over 45 min. Prior to biophysical or activity assays, peptides were purified to achieve a purity >95% by analytical rp-HPLC. The peptide concentration was assessed based on UV absorption at λ=280 nm measured on a NanoDrop 1000 spectrophotometer (Thermo Scientific) and extinction coefficient. Data is provided in Table 3.

TABLE 3
Analytical Characterization Data Confirming
Identity (LC-MS) and Purity (rp-HPLC)

| | | | | | | LCMS | | Retention |
|---|---|---|---|---|---|---|---|---|
| | | | | | LCMS | RT | HPLC | Time |
| Sample | Name | MW | M + 4/4 | M + 5/5 | Observd | (Min) | Purity | (Min) |
| 1 | SCI-a-KB29 | 6516.40 | 1630.10 | 1304.28 | Yes | 14.65 | >95% | 15.45 |
| 2 | SCI-a-KB28, PB29 | 6516.40 | 1630.10 | 1304.28 | Yes | 14.92 | >95% | 15.5 |
| 3 | SCI-a-RB29, KC5 | 6516.40 | 1630.10 | 1304.28 | Yes | 14.77 | >98% | 15.48 |
| 4 | SCI-a-KB3, RB29 | 6558.48 | 1640.62 | 1312.696 | Yes | 14.47 | >98% | 15.09 |
| 5 | SCI-a-AB0, RB29 | 6615.49 | 1654.87 | 1324.098 | Yes | 14.58 | >95% | 15.57 |
| 6 | SCI-a-KA14, RB29 | 6543.47 | 1636.87 | 1309.694 | Yes | 14.4 | >95% | 14.97 |
| 7 | SCI-a- | 6423.31 | 1606.83 | 1285.662 | TBD | TBD | | |
| | TA8, GA21, KB29 | | | | | | | |
| 8 | SCI-a- | 6810.40 | 1703.60 | 1363.08 | Yes | 22.52 | >98% | 24.12 |
| | K(C20)B28, PB29 | | | | | | | |
| 9 | SCI-a-RB29, | 6810.40 | 1703.60 | 1363.08 | Yes | 21.06 | >95% | 22.35 |
| | K(C20)C5 | | | | | | | |
| 10 | SCI-a-K(C20)B3, | 6852.48 | 1714.12 | 1371.496 | Yes | 21.58 | >95% | 22.43 |
| | RB29 | | | | | | | |
| 11 | SCI-a-K(C20B29 | 6810.40 | 1703.60 | 1363.08 | | | | |
| 12 | SCI-a-C20-AB0, | 6909.49 | 1728.37 | 1382.898 | | | | |
| | RB29 | | | | | | | |
| 13 | SCI-a- | 6837.47 | 1710.37 | 1368.494 | | | | |
| | K(C20)A14, RB29 | | | | | | | |
| 14 | SCI-a- | 6717.31 | 1680.33 | 1344.462 | | | | |
| | TA8,GA21, | | | | | | | |
| | K(C20)B29 | | | | | | | |

A method for treating a patient with diabetes mellitus or otherwise lowering the blood sugar of a patient in need thereof comprises administering a physiologically effective amount of a single-chain insulin analogue as described herein. It is another aspect of the present invention that the single-chain insulin analogues, such as those of SEQ ID NOS: 4-20, may be encoded by a nucleic acid and expressed and prepared in yeast, such as Pichia pastoris, or subject to total chemical synthesis by native fragment ligation. The synthetic route of preparation may be preferred in the case of non-standard modifications, such as D-amino-acid substitutions, halogen substitutions within the aromatic rings of Phe or Tyr, or O-linked modifications of Serine or Threonine by carbohydrates; however, it may also be feasible to manufacture a subset of the single-chain analogues containing non-standard modifications by means of extended genetic-code technology or four-base codon technology (for review, see Hohsaka, T., & Sisido, M., 2012). It is yet another aspect of the present invention that use of non-standard amino-acid substitutions can augment the resistance of the single-chain insulin analogue to chemical degradation or to physical degradation. We further envision the analogues of the present invention providing a method for the treatment of diabetes mellitus or the metabolic syndrome. The route of delivery of the insulin analogue is by subcutaneous injection through the use of a syringe or pen device.

A single-chain insulin analogue of the present invention may also contain other modifications, such as a halogen atom at positions B24, B25, or B26 as described more fully in co-pending U.S. Pat. No. 8,921,313, the disclosure of which is incorporated by reference herein. An insulin analogue of the present invention may also contain a foreshortened B-chain due to deletion of residues B1-B3 as described more fully in U.S. Pat. No. 9,725,493.

A pharmaceutical composition may comprise such insulin analogues and which may optionally include zinc. For acylated single-chain insulin analogues with isoelectric point in the range 4.0-6.0, clear and soluble formulations may be obtained as described in the art for insulin products Humalog and Levemir. Zinc ions may be included at varying zinc ion:protein ratios, ranging from 2.2 zinc atoms per insulin analogue hexamer to 10 zinc atoms per insulin analogue hexamer. Alternatively, the formulation may contain zinc ions at a molar ratio of between 2 and 10 zinc ions per six single-chain insulin analogue monomer. In another example, the formulation may contain zinc ions at a molar ratio of between 2 and 4 zinc ions per six single-chain insulin analogue monomer. Such a formulation may have a pH between 6.9 and 7.8.

For the class of acylated single-chain analogues with isoelectric point shifted to the range 6.5-7.5, the pH of the formulation is in the range pH 3.0-4.5. In such a formulation, the concentration of the insulin analogue would typically be between about 0.6-5.0 mM; concentrations up to 5 mM may be used in vial or pen; the more concentrated formulations (U-200 or higher) may be of particular benefit in patients with marked insulin resistance. Excipients may include glycerol, glycine, arginine, Tris, other buffers and salts, and anti-microbial preservatives such as phenol and meta-cresol; the latter preservatives are known to enhance the stability of the insulin hexamer. Such a pharmaceutical composition may be used to treat a patient having diabetes mellitus or other medical condition by administering a physiologically effective amount of the composition to the patient.

Based upon the foregoing disclosure, it should now be apparent that acylation of single-chain insulin analogues provided will carry out the objects set forth hereinabove. Namely, these insulin analogues combine enhanced resistance to fibrillation while conferring prolonged action relative to the unmodified single-chain parent analogues. Such modified analogues will maintain at least a fraction of the biological activity of wild-type insulin. It is, therefore, to be understood that any variations evident fall within the scope of the claimed invention and thus, the selection of specific component elements can be determined without departing from the spirit of the invention herein disclosed and described.

The following literature is cited to demonstrate that the testing and assay methods described herein would be understood by one of ordinary skill in the art.
Glendorf, T., Knudsen, L., Stidsen, C. E., Hansen, B. F., Hegelund, A. C., Sørensen, A. R., Nishimura, E., & Kjeldsen, T. 2012. Systematic evaluation of the metabolic to mitogenic potency ratio for B10-substituted insulin analogues. PLoS One 7(2), e29198.
Glidden, M. D., Aldabbagh, K., Phillips, N. B., Can, K., Chen, Y. S., Whittaker, J., Phillips, M., Wickramasinghe, N. P., Rege, N., Swain, M., Peng, Y., Yang, Y., Lawrence, M. C., Yee, V. C., Ismail-Beigi, F., & Weiss, M. A. (2018) An ultra-stable single-chain insulin analogue resists thermal inactivation and exhibits biological signaling duration equivalent to the native protein. J. Biol. Chem. 293(1), 47-68. PMIC5766902.
Glidden, M. D., Yang, Y., Smith, N. A., Phillips, N. B., Carr, K., Wickramasinghe, N. P., Ismail-Beigi, F., Lawrence, M. C., Smith, B. J., & Weiss, M. A. (2018) Solution structure of an ultra-stable single-chain insulin analog connects protein dynamics to a novel mechanism of receptor binding. J. Biol. Chem. 293(1), 69-88. PMIC5766920
Hohsaka, T., & Sisido, M. 2012. Incorporation of non-natural amino acids into proteins. Curr. Opin. Chem. Biol. 6, 809-15.
Hua, Q. X., Nakagawa, S. H., Jia, W., Huang, K., Phillips, N. B., Hu, S. & Weiss, M. A. (2008) Design of an active ultrastable single-chain insulin analog: synthesis, structure, and therapeutic implications. J. Biol. Chem. 283, 14703-14716.
Kristensen, C., Andersen, A. S., Hach, M., Wiberg, F. C., Schäffer, L., & Kjeldsen, T. 1995. A single-chain insulin-like growth factor I/insulin hybrid binds with high affinity to the insulin receptor. Biochem. J. 305, 981-6.
Lee, H. C., Kim, S. J., Kim, K. S., Shin, H. C., & Yoon, J. W. 2000. Remission in models of type 1 diabetes by gene therapy using a single-chain insulin analogue. Nature 408, 483-8. Retraction in: Lee H C, Kim K S, Shin H C. 2009. Nature 458, 600.
Phillips, N. B., Whittaker, J., Ismail-Beigi, F., & Weiss, M. A. (2012) Insulin fibrillation and protein design: topological resistance of single-chain analogues to thermal degradation with application to a pump reservoir. J. Diabetes Sci. Technol. 6, 277-288.

What is claimed is:

1. A single-chain insulin analogue comprising the insulin B-chain polypeptide sequence, the insulin A-chain polypeptide sequence, and a connecting polypeptide sequence of 5-11 amino acids linking the C-terminal amino acid of the B-chain polypeptide to the N-terminal amino acid of the A-chain polypeptide, wherein the single chain insulin analogue comprises an acetylated Lys at a location selected from the group consisting of any of the amino acids in the connecting polypeptide, B0-B3, B28-B29 or A14, relative to wild type insulin, or comprising an acetylated amino acid at the N-terminal amino acid of the single-chain insulin analogue.

2. The single-chain insulin analogue of claim 1, wherein the single-chain insulin analogue is acylated with a C₆-C₂₁ fatty acid.

3. The single-chain insulin analogue of claim 2, wherein the C₆-C₂₁ fatty acid is selected from the group consisting of myristic acid, pentadecylic acid, palmitic acid, heptadecylic acid, steric acid and arachidic acid.

4. The single-chain insulin analogue of claim 3, wherein the C₆-C₂₁ fatty acid is selected from the group consisting of palmitic acid and arachidic acid.

5. The single-chain insulin analogue of claim 4, comprising an amino acid sequence selected from the group consisting of SEQ ID NOS: 4, 5, and 14-20.

6. The single-chain insulin analogue of claim 2, wherein the C₆-C₂₁ fatty acid is attached to the ε-amino group of a unique Lysine residue in the A-chain polypeptide, the B-chain polypeptide or the connecting peptide.

7. The single-chain insulin analogue of claim 6, wherein the C₆-C₂₁ fatty acid is selected from the group consisting of myristic acid, pentadecylic acid, palmitic acid, heptadecylic acid, steric acid and arachidic acid.

8. The single-chain insulin analogue of claim 7, wherein the C₆-C₂₁ fatty acid is selected from the group consisting of palmitic acid and arachidic acid.

9. The single-chain insulin analogue of claim 2 wherein the C₆-C₂₁ fatty acid is attached to the α-amino group of the N-terminal amino acid of the single-chain insulin analogue.

10. The single-chain insulin analogue of claim 9, wherein the N-terminal amino acid is Ala.

11. The single-chain insulin analogue of claim 10, wherein the C₆-C₂₁ fatty acid is selected from the group consisting of myristic acid, pentadecylic acid, palmitic acid, heptadecylic acid, steric acid and arachidic acid.

12. (canceled)

13. A method of lowering the blood sugar level of a patient in need thereof, the method comprising administering a physiologically effective amount of a single-chain insulin analogue or a physiologically acceptable salt thereof to the patient, wherein the single-chain insulin analogue comprises the insulin B-chain polypeptide sequence, the insulin A-chain polypeptide sequence, and a connecting polypeptide sequence of 5-11 amino acids linking the C-terminal amino acid of the B-chain polypeptide to the N-terminal amino acid of the A-chain polypeptide, wherein the single chain insulin analogue comprises an acetylated Lys at a location selected from the group consisting of any of the amino acids in the connecting polypeptide, B0-B3, B28-B29 or A14, relative to wild type insulin, or comprising an acetylated amino acid at the N-terminal amino acid of the single-chain insulin analogue.

14. The method of claim 13, wherein the single-chain insulin analogue is acylated with a C₆-C₂₁ fatty acid.

15. The method of claim 14, wherein the C₆-C₂₁ fatty acid is selected from the group consisting of myristic acid, pentadecylic acid, palmitic acid, heptadecylic acid, steric acid and arachidic acid.

16. The method of claim 15, wherein the single-chain insulin analogue comprises an amino acid sequence selected from the group consisting of SEQ ID NOS: 4, 5 and 14-20.

17. A nucleic acid encoding the polypeptide of any one of SEQ ID NOS: 4-13.

18. The single-chain insulin analogue of claim 1, additionally comprising zinc ions at a molar ratio between 2 and 4 zinc ions per six single-chain insulin analogue monomers to provide a pharmaceutical formulation.

19. The single-chain insulin analogue of claim 18, wherein the pH of the formulation is between pH 3.0 and 4.5.

20. The single-chain insulin analogue of claim 18, wherein the pH of the formulation is between pH 6.5 and 7.8.

21. The single-chain insulin analogue of claim 4, wherein the connecting peptide consists of the amino acid sequence of residues 31-36 of SEQ ID NO: 4.


SEQUENCE LISTING (23 sequences)

SEQ ID NO: 1 - protein, 86 aa, Homo sapiens
FVNQHLCGSHLVEALYLVCGERGFFYTPKTRREAEDLQVGQVELGGGPGAGSLQPLALEGSLQKRGIVEQCCTSICSLYQLENYCN

SEQ ID NO: 2 - protein, 21 aa, Homo sapiens
GIVEQCCTSICSLYQLENYCN

SEQ ID NO: 3 - protein, 30 aa, Homo sapiens
FVNQHLCGSHLVEALYLVCGERGFFYTPKT

SEQ ID NO: 4 - protein, 57 aa, Artificial Sequence
  SYNTHETIC CONSTRUCT
  MISC_FEATURE (35)..(35): Xaa is Ala, Glu or Arg
  MISC_FEATURE (44)..(44): Xaa is Ala, Glu, Gln, His, or Thr
  MISC_FEATURE (50)..(50): Xaa is Tyr or Glu
  MISC_FEATURE (57)..(57): Xaa is Gly, Ala, Asn, Ser or Thr
FVNQHLCGSHLVEALYLVCGERGFFYTPRTEEGPXRGIVEQCCXSICSLXQLENYCX

SEQ ID NO: 5 - protein, 63 aa, Artificial Sequence
  SYNTHETIC CONSTRUCT
  MISC_FEATURE (1)..(1): Xaa is Ala, Gln or is absent
  MISC_FEATURE (2)..(2): Xaa is Ala, Phe or is absent
  MISC_FEATURE (3)..(3): Xaa is Ala, Val or is absent
  MISC_FEATURE (4)..(4): Xaa is Ala, Asn or Glu
  MISC_FEATURE (30)..(30): Xaa is Ala, Glu or Arg
  MISC_FEATURE (32)..(42): Xaa is 5-11 amino acids where at least one of residues 32 and 33 is Glu and where the final two residues in the segment contain at least one Arg
  MISC_FEATURE (50)..(50): Xaa is Ala, Glu, Gln, His, or Thr
  MISC_FEATURE (56)..(56): Xaa is Tyr or Glu
  MISC_FEATURE (63)..(63): Xaa is Gly, Ala, Asn, Ser or Thr
XXXXQHLCGSHLVEALYLVCGERGFFYTPXTXXXXXXXXXXXGIVEQCCXSICSLXQLENYCX

SEQ ID NO: 6 - protein, 63 aa, Artificial Sequence
  SYNTHETIC CONSTRUCT
  MISC_FEATURE (1)..(1): Xaa is Ala, Glu or is absent
  MISC_FEATURE (2)..(2): Xaa is Ala, Phe or is absent
  MISC_FEATURE (3)..(3): Xaa is Ala, Val or is absent
  MISC_FEATURE (4)..(4): Xaa is Ala, Asn or Glu
  MISC_FEATURE (29)..(30): Xaa at 29 is Pro, Ala, Asp, Glu or optionally the choice of a unique Lys and Xaa at 30 is Ala, Glu, Arg, Pro or optionally a unique Lys as in wildtype insulin; provided that the amino acides at positions 29 and 30 are not both Pro
  MISC_FEATURE (32)..(42): Xaa is 5-11 amino acids where at least one of residues 32 or 33 is Glu, where the final two residues in the segment contain at least one Arg and optionally containing a unique Lysine
  MISC_FEATURE (50)..(50): Xaa is Ala, Glu, Gln, His, or Thr
  MISC_FEATURE (56)..(56): Xaa is Tyr or Glu
  MISC_FEATURE (63)..(63): Xaa is Gly, Ala, Asn, Ser or Thr
XXXXQHLCGSHLVEALYLVCGERGFFYTXXTXXXXXXXXXXXGIVEQCCXSICSLXQLENYCX

SEQ ID NO: 7 - protein, 57 aa, Artificial Sequence
  SYNTHETIC CONSTRUCT
FVNQHLCGSHLVEALYLVCGERGFFYTPKTEEGPRRGIVEQCCHSICSLEQLENYCN

SEQ ID NO: 8 - protein, 57 aa, Artificial Sequence
  SYNTHETIC CONSTRUCT
FVNQHLCGSHLVEALYLVCGERGFFYTKPTEEGPRRGIVEQCCHSICSLEQLENYCN

SEQ ID NO: 9 - protein, 57 aa, Artificial Sequence
  SYNTHETIC CONSTRUCT
FVNQHLCGSHLVEALYLVCGERGFFYTPRTEEGPKRGIVEQCCHSICSLEQLENYCN

SEQ ID NO: 10 - protein, 57 aa, Artificial Sequence
  SYNTHETIC CONSTRUCT
FVKQHLCGSHLVEALYLVCGERGFFYTPRTEEGPRRGIVEQCCHSICSLEQLENYCN

SEQ ID NO: 11 - protein, 58 aa, Artificial Sequence
  SYNTHETIC CONSTRUCT
AFVNQHLCGSHLVEALYLVCGERGFFYTPRTEEGPRRGIVEQCCHSICSLEQLENYCN

SEQ ID NO: 12 - protein, 57 aa, Artificial Sequence
  SYNTHETIC CONSTRUCT
FVNQHLCGSHLVEALYLVCGERGFFYTPRTEEGPRRGIVEQCCHSICSLKQLENYCN

SEQ ID NO: 13 - protein, 57 aa, Artificial Sequence
  SYNTHETIC CONSTRUCT
FVNQHLCGSHLVEALYLVCGERGFFYTPKTEEGPRRGIVEQCCTSICSLEQLENYCG

SEQ ID NO: 14 - protein, 57 aa, Artificial Sequence
  SYNTHETIC CONSTRUCT
  MISC_FEATURE (28)..(28): Xaa is acetylated Lys
FVNQHLCGSHLVEALYLVCGERGFFYTXPTEEGPRRGIVEQCCHSICSLEQLENYCN

SEQ ID NO: 15 - protein, 57 aa, Artificial Sequence
  SYNTHETIC CONSTRUC
  MISC_FEATURE (35)..(35): Xaa is acetylated Lys
FVNQHLCGSHLVEALYLVCGERGFFYTPRTEEGPXRGIVEQCCHSICSLEQLENYCN

SEQ ID NO: 16 - protein, 57 aa, Artificial Sequence
  SYNTHETIC CONSTRUCT
  MISC_FEATURE (3)..(3): Xaa is acetylated Lys
FVXQHLCGSHLVEALYLVCGERGFFYTPRTEEGPRRGIVEQCCHSICSLEQLENYCN

SEQ ID NO: 17 - protein, 57 aa, Artificial Sequence
  SYNTHETIC CONSTRUCT
  MISC_FEATURE (29)..(29): Xaa is acetylated Lys
FVNQHLCGSHLVEALYLVCGERGFFYTPXTEEGPRRGIVEQCCHSICSLEQLENYCN

SEQ ID NO: 18 - protein, 58 aa, Artificial Sequence
  SYNTHETIC CONSTRUCT
  MISC_FEATURE (1)..(1): Xaa is acetylated Ala
XFVNQHLCGSHLVEALYLVCGERGFFYTPRTEEGPRRGIVEQCCHSICSLEQLENYCN

SEQ ID NO: 19 - protein, 57 aa, Artificial Sequence
  SYNTHETIC CONSTRUCT
  MISC_FEATURE (50)..(50): Xaa is acetylated Lys
FVNQHLCGSHLVEALYLVCGERGFFYTPRTEEGPRRGIVEQCCHSICSLXQLENYCN

SEQ ID NO: 20 - protein, 56 aa, Artificial Sequence
  SYNTHETIC CONSTRUC
  MISC_FEATURE (28)..(28): Xaa is acetylated Lys
VNQHLCGSHLVEALYLVCGERGFFYTPXTEEGPRRGIVEQCCTSICSLEQLENYCG

SEQ ID NO: 21 - protein, 11 aa, Artificial Sequence
  SYNTHETIC CONSTRUCT
  MISC_FEATURE (2)..(10): where Xaa from positions 2-10 is 3-9 of any amino acid
EXXXXXXXXXR

SEQ ID NO: 22 - protein, 11 aa, Artificial Sequence
  SYNTHETIC CONSTRUCT
  MISC_FEATURE (2)..(10): where Xaa from positions 2-10 is 3-9 of any amino acid
EXXXXXXXXXA

SEQ ID NO: 23 - protein, 11 aa, Artificial Sequence
  SYNTHETIC CONSTRUCT
  MISC_FEATURE (2)..(10): where Xaa from positions 2-10 is 3-9 of any amino acid
EXXXXXXXXXS